(19) United States
(12) Patent Application Publication
(10) Pub. No.: US 2025/0319613 A1
(43) Pub. Date: Oct. 16, 2025

(54) ROBOTIC HANDS AND METHODS OF USING A ROBOTIC HAND

(71) Applicant: Purdue Research Foundation, West Lafayette, IN (US)

(72) Inventors: Haiyan Henry Zhang, West Lafayette, IN (US); Heming Liu, West Lafayette, IN (US)

(21) Appl. No.: 19/179,113

(22) Filed: Apr. 15, 2025

Related U.S. Application Data
(60) Provisional application No. 63/634,186, filed on Apr. 15, 2024.

(51) Int. Cl. B25J 15/10; A61F 2/50; A61F 2/58

(52) U.S. Cl. CPC B25J 15/10; A61F 2/586; A61F 2002/5038; A61F 2002/587

(57) ABSTRACT

Robotic hands and methods of using a robotic hand. Such a robotic hand has a palm frame that defines a palm plane and a plurality of articulating fingers carried by the palm frame. The size and orientation of the palm frame may be adjusted within the palm plane. The articulating fingers can articulate toward both opposite sides of the palm plane such that the robotic hand can be oriented in either a right hand configuration or a left hand configuration.

### BACKGROUND OF THE INVENTION

This application claims the benefit of provisional U.S. Patent Application No. 63/634,186 filed Apr. 15, 2024, the contents of which are incorporated herein by reference.

### BACKGROUND OF THE INVENTION

The invention generally relates to a robotic hand and methods of using the robotic hand.

Conventional robotic hands, sometimes referred to as metamorphic or prosthetic hands, are typically configured for use in a single size or orientation. Thus, different use applications typically require different types of robotic hands, which can increase costs and decrease the versatility. Therefore, it would be desirable to have a robotic hand that can be used for a variety of different use scenarios with different sizes and orientations.

### BRIEF SUMMARY OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

The present invention provides, but is not limited to, robotic hands and methods of using robotic hands.

According to a nonlimiting aspect, a robotic hand includes a palm frame and a plurality of articulating fingers. The palm frame includes a first linear actuator having a first component and a second component that is linearly shiftable relative to the first component along a first actuation axis, a second linear actuator having a third component and a fourth component that is linearly shiftable relative to the first component along a second actuation axis, a third linear actuator having a fifth component and a sixth component that is linearly shiftable relative to the first component along a third actuation axis, and a link having a first end and a second end defining a fourth axis. The second component is rotatably connected to the third component about a first rotational axis, the fourth component is rotatably connected to the fifth component about a second rotational axis, the sixth component is rotatably connected to the first end of the link about a third rotational axis, and the second end of the link is rotatably connected to the first component about a fourth rotational axis. Each of the first axis, the second axis, the third axis, and the fourth axis are parallel with each other. The articulating fingers are coupled to the second and third linear actuators of the palm frame. Extending and/or retracting any one or more of the first linear actuator, the second linear actuator, and the third linear actuator changes the size of the palm within a palm plane and/or one or more angles about the first, second, and third rotational axes.

According to another nonlimiting aspect, a method of using the above-described robotic hand includes changing the size of the palm and/or one or more angles about the first, second, and third axes by extending and/or retracting any one or more of the first linear actuator, the second linear actuator, and the third linear actuator.

According to yet another nonlimiting aspect, a robotic hand includes a palm frame and a plurality of articulating fingers coupled to the palm frame. Each of the articulating fingers can articulate in a first direction toward a first side of the palm plane and in a second direction toward a second side of the palm plane.

According to still another nonlimiting aspect, a method of using the robotic hand described above includes articulating the articulating fingers together toward a first side of the palm plane and articulating the fingers together toward a second side of the palm plane.

Technical aspects of robotic hands and associated methods as described above preferably include the ability to adapt to both left hand and right hand orientations and/or adapt in size and orientation to accommodate improved grasping objects of different shapes and sizes.

These and other aspects, arrangements, features, and/or technical effects will become apparent upon detailed inspection of the figures and the following description.

### BRIEF DESCRIPTION OF THE DRAWINGS

FIG. 1 is an isometric view of a robotic hand with five articulating fingers (digits) according to a nonlimiting embodiment of the invention.

FIGS. 2A and 2B are isometric and side views, respectively, of components of a palm of the robotic hand of FIG. 1.

FIG. 3 is an isometric view of components of one of the articulating fingers of the robotic hand of FIG. 1 operatively coupled to a portion of the palm.

FIG. 4 illustrates five planes of operation of the five articulating fingers of the robotic hand of FIG. 1.

FIGS. 5 through 19 represent various rotation actuation mechanisms by which articulation of the articulating fingers may be controlled according to nonlimiting aspects of the invention.

### DETAILED DESCRIPTION OF THE INVENTION

The intended purpose of the following detailed description of the invention and the phraseology and terminology employed therein is to describe what is shown in the drawings, which include the depiction of and/or relate to one or more nonlimiting embodiments of the invention, and to describe certain but not all aspects of the embodiment(s) depicted in the drawings. The following detailed description also identifies certain but not all alternatives of the embodiment(s) depicted in the drawings. As nonlimiting examples, the invention encompasses additional or alternative embodiments in which one or more features or aspects shown and/or described as part of a particular embodiment could be eliminated, and also encompasses additional or alternative embodiments that combine two or more features or aspects described as part of different embodiments. Therefore, the appended claims, and not the detailed description, are intended to particularly point out subject matter regarded as aspects of the invention, including certain but not necessarily all of the aspects and alternatives described in the detailed description.

The drawings represent various aspects of a robotic (metamorphic or prosthetic) hand 10 that is preferably capable of adapting to both left hand and right hand orientations, and adapting in size and orientation to accommodate improved grasping objects of different shapes and sizes. To facilitate the description provided below of the embodiment(s) represented in the drawings, relative terms, including but not limited to, “proximal,” “distal,” “anterior,” “posterior,” “vertical,” “horizontal,” “lateral,” “front,” “rear,” “side,” “forward,” “rearward,” “top,” “bottom,” “upper,” “lower,” “above,” “below,” “right,” “left,” etc., may be used in reference to the orientation of the robotic hand 10 during its use and/or as represented in the drawings. All such relative terms are useful to describe the illustrated embodiment(s) but should not be otherwise interpreted as limiting the scope of the invention.

As used herein the terms “a” and “an” to introduce a feature are used as open-ended, inclusive terms to refer to at least one, or one or more of the features, and are not limited to only one such feature unless otherwise expressly indicated. Similarly, use of the term “the” in reference to a feature previously introduced using the term “a” or “an” does not thereafter limit the feature to only a single instance of such feature unless otherwise expressly indicated.

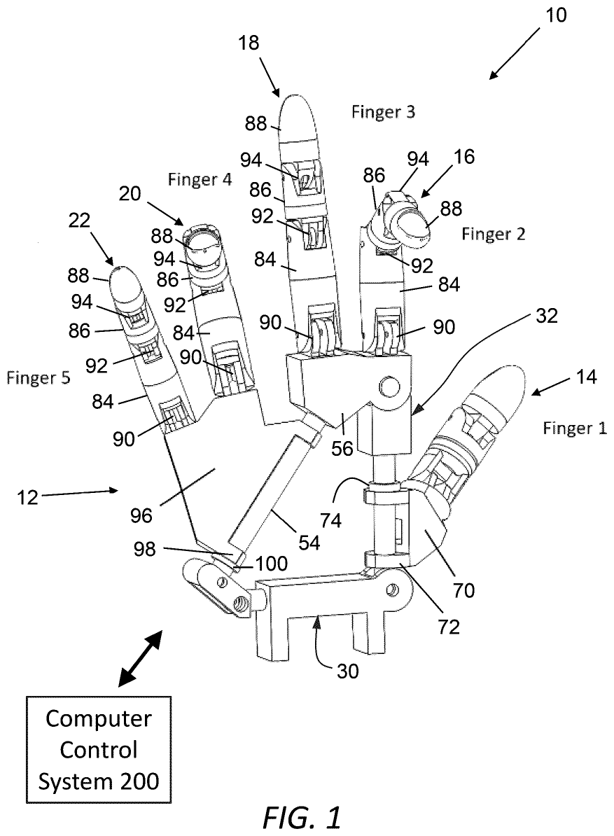

Turning now to the nonlimiting embodiments represented in the drawings, FIG. 1 depicts the robotic hand 10 as comprising five articulating digits 14, 16, 18, 20, and 22 representative of four fingers and a thumb of a human hand. The digits 14, 16, 18, 20, and 22 are referred to herein as fingers (collectively, fingers 14-22) as a matter of convenience, and as such the hand 10 may be referred to herein as a five-finger device. The robotic hand 10 includes a robotic palm structure comprising a frame 12 (“palm frame”), to which the five articulating fingers 14-22 are connected or interconnected. In the depicted embodiment, the five fingers 14-22 correspond to, respectively, the thumb, index finger (second digit), middle finger (third digit), ring finger (fourth digit), and little (“pinky”) finger (fifth digit) of a typical human hand, and therefore will be individually identified as such in the following discussion, i.e., the thumb 14, index finger 16, middle finger 18, ring finger 20, and pinky finger 22. Other configurations of the articulating fingers 14-22 could be implemented. Although the embodiment shown in FIG. 1 has five fingers 14-22 to simulate a typical human hand, fewer or more articulating fingers could be implemented.

The palm frame 12 is a closed-loop series structure made of multiple components that are represented as including four rotary joints and three prismatic joints. By controlling the configuration of the rotary joints and the prismatic joints, the palm frame 12 can be scaled (larger and smaller) and deformed (change of shape) to accommodate any of many different needed configurations for various different use scenarios. This adjustability of the palm frame 12 is convenient to allow the articulating fingers 14-22 to achieve the best enveloping grasp effect when grabbing objects of various form factors, and thereby increasing the stability of the grasp.

The embodiment represented in FIG. 1 depicts the articulating fingers 14-22 as comprising finger joints (knuckles) 90, 92, and 94 between a proximal link 84 of each finger 14-22 and the palm frame 12, between the proximal link 84 and a medial link 86 of each finger 14-22, and between the medial link 86 and a distal link 88 of each finger 14-22. In the embodiment shown, the proximal, medial, and distal links 84, 86, and 88 generally correspond to the proximal, intermediate, and distal phalanges of a human hand, in which case the finger joints 90 between the palm frame 12 and proximal link 84 of each finger 14-22 may be described as generally corresponding to the metacarpophalangeal joints of the fingers 14-22 and the finger joints 92 and 94 between the proximal, medial, and distal links 84, 86, and 88 of each finger 14-22 may be described as generally corresponding to the interphalangeal joints of the fingers 14-22. The finger joints 90, 92, and 94 are represented as comprising pinned connections such that each is configured to have bi-directional rotation, whereby each of the links 84, 86, and 88 can pivot about its corresponding finger joint 90, 92, and/or 94 toward either side of the palm frame 12 (e.g., in both a “positive” angular direction and a “negative” angular direction relative to a plane defined by the palm frame 12). This allows the robotic hand 10 to have a reconfigurable, bi-directional grasping capability, which can function as either/or both a left hand and/or a right hand of various sizes.

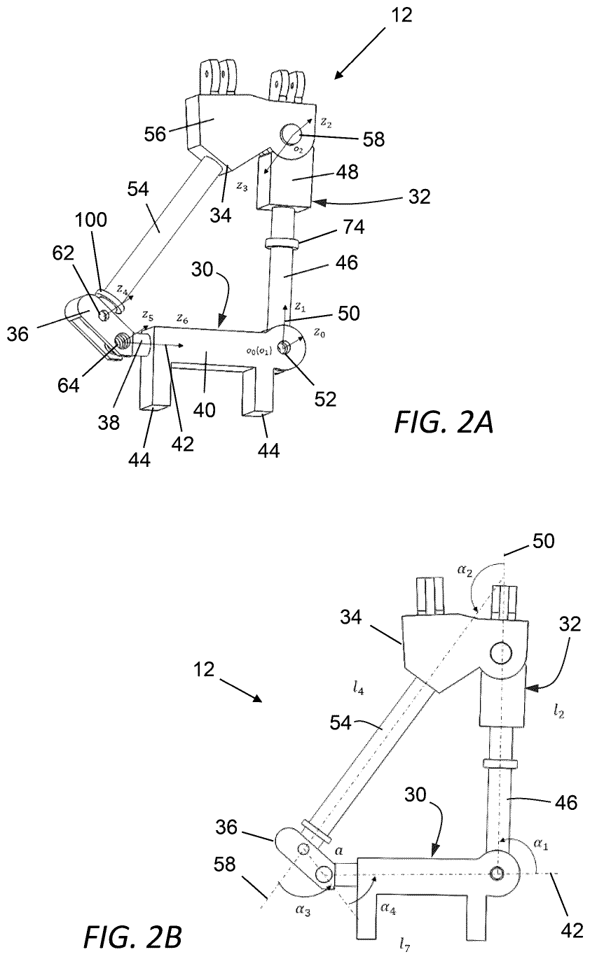

As best seen in FIG. 2A and 2B, the palm frame 12 has a first linear actuator 30, a second linear actuator 32, a third linear actuator 34, and a link 36 having a fixed length. The first linear actuator 30 has a tip part 38 and an end part 40 which can move linearly relative to each other along a first actuation axis 42. The end part 40 forms the base of the robotic hand 10 and includes one or more connector protrusions 44 that are adapted to connect with an external support structure to control the pitch angle of the entire robotic hand 10. The second linear actuator 32 has a tip part 46 and an end part 48 that move linearly relative to each other along a second actuation axis 50. The tip part 46 is linked to the end part 40 by a rotational connection 52, such as a pin and hinge connection (“rotary pair”). The third linear actuator 34 has tip part 54 and end part 56 that move linearly relative to each other along a third actuation axis 58. The end part 56 forms a base of the index finger 16 and middle finger 18. A rotational connection 58 (e.g., a pin and hinge “rotary pair”) connects the end part 48 of the second linear actuator 32 to the end part 56 of the linear actuator component 34. The link 36 connects to tip part 54 of the third linear actuator 34 to the tip part 38 of the first linear actuator 30 with two more rotary pairs formed by a rotational connection 62 that connects the tip part 54 to one end of the link 36, and a rotational connection 64 that connects the tip part 38 to the other end of the link 36. The palm frame 12 lies substantially in a single plane that defines a palm plane. Each of the rotational connections 52, 58, 62, and 64 rotates about an axis that is substantially perpendicular to the palm plane such that all of the rotational axes are substantially parallel with each other.

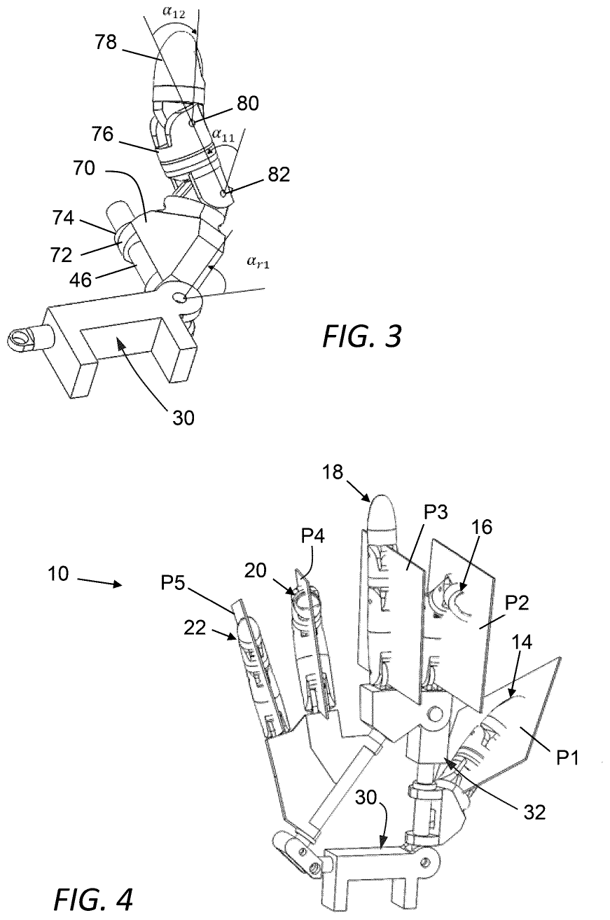

As depicted in FIGS. 1 and 3, the thumb 14 has a thumb base structure 70 that is connected to the tip part 46 of the second linear actuator 32 by a rotary pair 72, which in this example is a pair of collars rotatably mounted around a cylindrical body of the tip part 46. Axial movement of the thumb base structure 70 along tip part 46 is restricted by a shoulder structure 74 on the tip part 46 and the base 40. The thumb base component 70 can rotate around the axis 50 of the second linear actuator 32. The plane of the thumb base component and the plane of the palm frame 12 can rotate relative to each other to have a relative angle range of −60° to +60°. The thumb 14 forms a jointed finger having two articulating links, a thumb proximal link 76 and a thumb distal link 78, pivotably coupled by a pivot joint 80. The proximal link 76 is also pivotably connected to the thumb base component 70 by a pivot joint 82. The axis of the thumb base component 70 and the axis of thumb proximal link 76 can articulate a relative angle range of −80° to +80°. The axis of the thumb proximal link 76 and the axis of thumb distal link 78 can articulate a relative angle range of −90° to +90°. The thumb distal link 78 and the thumb proximal link 76 articulate in a single plane, the thumb plane, which is disposed at an acute angle relative to the second actuation axis 50.

As discussed above, FIG. 1 represents palm frame 12 and the proximal, medial, and distal links 84, 86, and 88 of each of the index finger 16, middle finger 18, ring finger 20, and pinky finger 22 as pivotably connected together by the finger joints 90, 92, and 94 to enable each finger 16, 18, 20, and 22 to articulate in a (different) single plane. The proximal link 84, medial link 86, and distal link 88 may have the same lengths or different lengths. In this embodiment, the proximal link 84, medial link 86, and distal link 88 of each finger 16-22 is shaped to generally have the same shape and length as human fingers, which would be useful when used as a human prosthetic device, for example. However, other shapes and sizes of the finger links could be implemented for other uses.

Each of the index finger 16 and the middle finger 18 is mounted to the end part 56 of the third actuator 34 so as to be capable of articulation. The index finger 16 and the middle finger 18 are nearly, although not exactly parallel with each other. In this example, the index finger 16 and the middle finger 18 are angularly offset from parallel a small amount (e.g., less than about 10° or less than about) 5°0 to form a slight V-shape. However, the index finger 16 and the middle finger 18 could be parallel with each other or other angles could be implemented in other embodiments. The proximal end of the proximal link 84 of each of the index finger 16 and the middle finger 18 is pivotably connected to the end part 56 of the third linear actuator 34 by the finger joint 90 therebetween, the distal end of the proximal link 84 and the proximal end of the medial link 86 are connected by the finger joint 92 therebetween, and the distal end of the medial link 86 and the proximal end of the distal link 88 are connected by the finger joint 94 therebetween. For each of the index finger 16 and the middle finger 18, the plane of the palm frame 12 and the axis of the proximal link 84 articulate through relative angle range of −80° to +80°, and the axes of the proximal link 84, medial link 86, and distal link 88 all articulate relative to each other an angle range of −90° to +90°.

The ring finger 20 and the pinky finger 22 are carried by a finger base 96 that is connected to the tip part 54 of the third linear actuator 34 by a rotary pair 98, which in this example is a pair of collars rotatably mounted around a cylindrical body of the tip part 54. Axial movement of the finger base 96 along tip part 54 is restricted by a shoulder structure 100 on the tip part 54 and the end part 56. The finger base 96 can rotate around the axis 58 of the third linear actuator 34. The plane of the finger base 96 and the plane of the palm frame 12 can rotate relative to each other to have a relative angle range of −30° to +30°. In this way, each of the ring finger 20 and the pinky finger 22 is mounted to the tip part 54 by the finger base 96 so as to be capable of articulation.

For each of the ring finger 20 and the pinky finger 22, the proximal end of the proximal link 84 is pivotably connected to the finger base 96 by the associated finger joint 90 therebetween, the distal end of the proximal link 84 and the proximal end of the medial link 86 are connected by the finger joint 92 therebetween, and the distal end of the medial link 86 and the proximal end of the distal link 88 are connected by the finger joint 94 therebetween. For each of the ring finger 20 and the pinky finger 22, the plane of the palm frame 12 and the axis of the proximal link 84 articulate through a relative angle range of −80° to +80°, and the axes of the index finger proximal link 84, medial link 86, and distal link 88 all articulate relative to each other an angle range of −90° to +90°. Similar to the index finger 16 and the middle finger 18, the ring finger 20 and the pinky finger 22 are nearly, although not exactly parallel with each other. In this example, the ring finger 20 and the pinky finger 22 are angularly offset from parallel a small amount (e.g., less than about 10° or less than about 5° to form a slight V-shape. However, the ring finger 20 and the pinky finger 22 could be parallel with each other or other angles could be implemented in other embodiments.

As depicted in FIG. 4, each of the five articulating fingers 14-22 articulates in a different plane about its respective finger joint 90: the thumb 14 articulates in the plane P1, the index finger 16 articulates in the plane P2, the middle finger 18 articulates in the plane P3, the ring finger 20 articulates in the plane P4, and the pinky finger 22 articulates in the plane P5. The plane P1 is disposed at an acute angle relative to the second actuation axis 50. Thus, the thumb 14 can both articulate about the second actuation axis 50 and articulate in the plane P1. The plane P2 is substantially parallel with (e.g., within about 10°) the second actuation axis 50. For example, the second actuation axis 50 may lie in the plane P2. The plane P3 is also substantially parallel with (e.g., within about 1020 ) the second actuation axis 50. The plane P3 is also offset laterally from the plane P2 at least at the location of the end piece 56. The plane P4 is disposed at an acute angle relative to the third actuation axis 58, and the plane P5 is disposed at another acute angle relative to the third actuation axis 58. As with the thumb, each of the ring finger 20 and the pinky finger 22 articulates about the third actuation axis 58 and also articulates within their respective planes P4 and P5. When each articulating finger 14-22 is fully extended, the fingers point generally in the same direction within a total angle sweep of the planes P1-P5 of less than about 90° thereby providing a clear left or right hand orientation depending which direction the fingers 14-22 articulate. Thus, for example, if the fingers 14-22 articulate in the direction out of the view toward the visible side of the palm plane as seen in FIGS. 1 and 4, the robotic hand 10 would function as a right hand. However, if the fingers 14-22 articulate in the direction into the view toward the non-visible side of the palm plane as seen in FIGS. 1 and 4, the robotic hand 10 would function as a left hand.

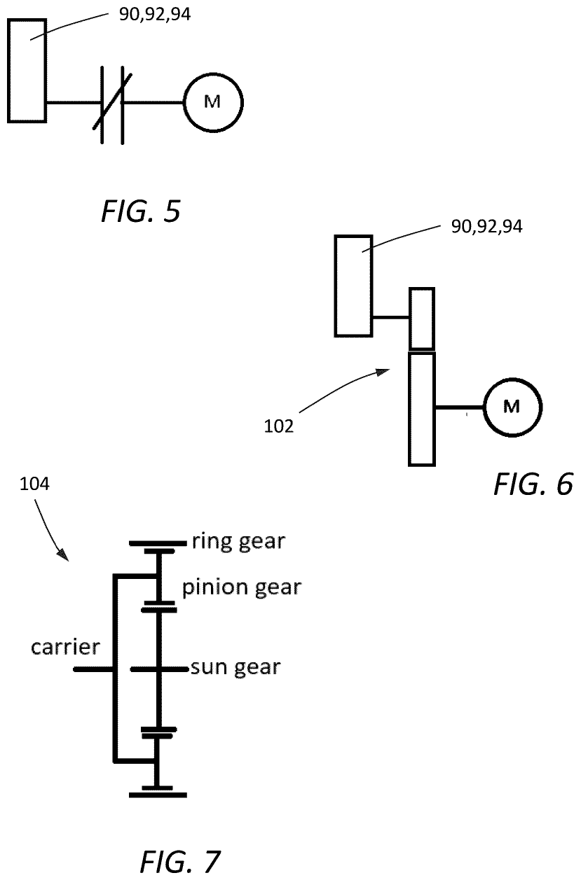

Control of the robotic hand 10 may be provided by any combination of hardware and/or software suitable to control movement of the linear actuators 30, 32, and 34 and control articulation of the articulating fingers 14-22. As a nonlimiting example, the linear actuators 30, 32, and 34 may be any one of a hydraulic actuator, a pneumatic actuator, a rack and pinion actuator, a servo motor, and combinations thereof. Control of the articulation of individual articulating fingers 14-22 may be provided by any suitable types of rotation actuation mechanisms that are preferably configured to individually actuate and rotate the proximal, medial, and distal links 84, 86, and 88 of each individual articulating fingers 14-22 at each of the finger joints 90, 92, and 94. Such mechanisms may include, for example, hydraulic actuators, pneumatic actuators, geared actuators, servo motors, and/or linear actuators. FIGS. 5 through 19 diagrammatically represent examples of such mechanisms utilized in combination with a DC drive motor (“M”).

FIG. 5 represents the drive motor M directly connected to a finger joint 90, 92, or 94 of one of the articulating fingers 14-22, and FIG. 6 represents the drive motor M connected to a finger joint 90, 92, or 94 of one of the articulating fingers 14-22 through a gear reduction 102.

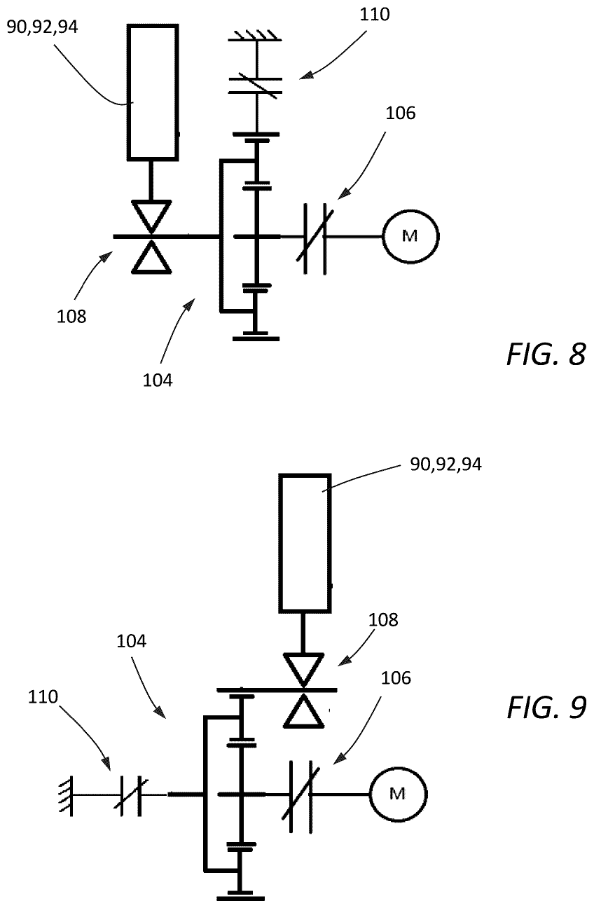

FIG. 7 schematically represents a planetary gear system 104 capable of being used as a component of a rotation actuation mechanism to articulate the finger joints 90, 92, and/or 94 of the fingers 14-22. As nonlimiting examples, FIGS. 8 through 19 represent embodiments in which the drive motor M is connected to a finger joint 90, 92, or 94 of one of the articulating fingers 14-22 through the planetary gear system 104 of FIG. 7. As known, the planetary gear system 104 is represented in FIG. 7 as comprising a sun gear, a carrier that is coaxial with the sun gear and to which pinion (planet) gears are mounted, and a ring gear that is coaxial with both the sun gear and carrier so that the pinion gears mesh with both the sun and ring gears. In order to provide more gear ratio choices than the planetary gear system 104, a Simpson planetary gearset may be utilized (not shown) in the embodiments of FIGS. 8 through 19 in place of the planetary gear system 104.

In FIG. 8, the drive motor M is directly connected to the sun gear of the planetary gear system 104 through a rotating mechanical clutch 106, and the carrier of the planetary gear system 104 is connected to the finger joint 90, 92, or 94 through a magnetic clutch 108 while the ring gear is connected to a base through a stationary clutch 110.

FIG. 9 shows the drive motor M directly connected to the sun gear through a rotating mechanical clutch 106, and the carrier is connected to a base through a stationary clutch 110, while the ring gear is connected to the finger joint 90, 92, or 94 through a magnetic clutch 108.

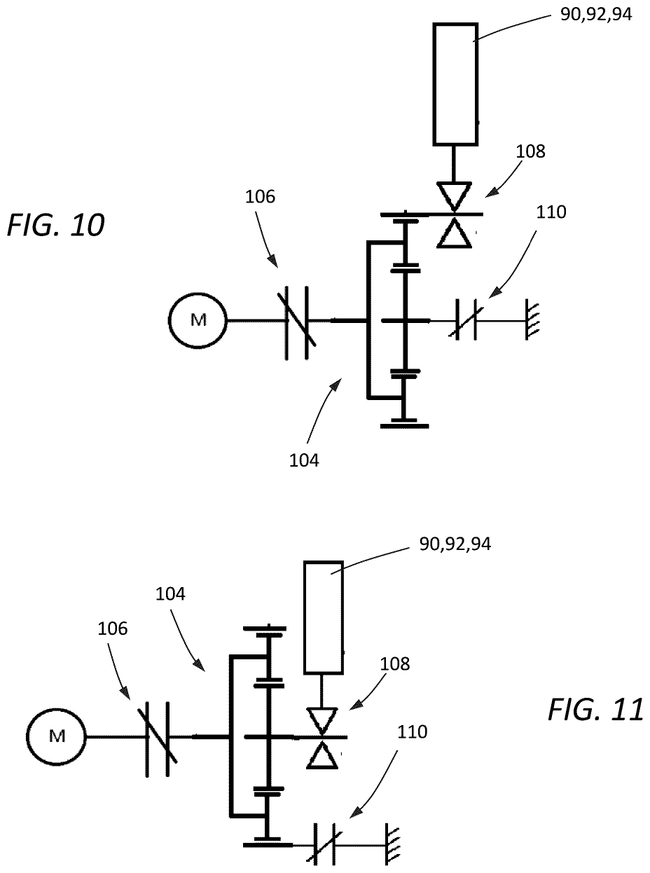

FIG. 10 shows the drive motor M directly connected to the carrier through a rotating mechanical clutch 106, and the sun gear connected to a base through a stationary clutch 110, while the ring gear is connected to the finger joint 90, 92, or 94 through a magnetic clutch 108.

In FIG. 11, the drive motor M is directly connected to the carrier through a rotating mechanical clutch 106, the sun gear is connected to the finger joint 90, 92, or 94 through a magnetic clutch 108, and the ring gear is connected to a base through a stationary clutch 110.

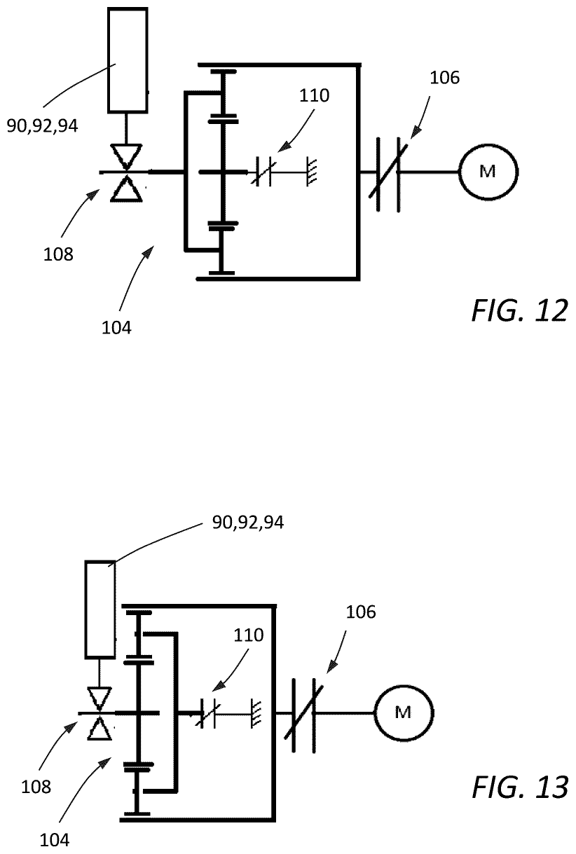

FIG. 12 shows the drive motor M directly connected to the ring gear through a rotating mechanical clutch 106, the sun gear connected to a base through a stationary clutch 110, and the carrier connected to the finger joint 90, 92, or 94 through a magnetic clutch 108.

FIG. 13 shows the drive motor M directly connected to the ring gear through a rotating mechanical clutch 106, the sun gear connected to the finger joint 90, 92, or 94 through a magnetic clutch 108, and the carrier connected to a base through a stationary clutch 110.

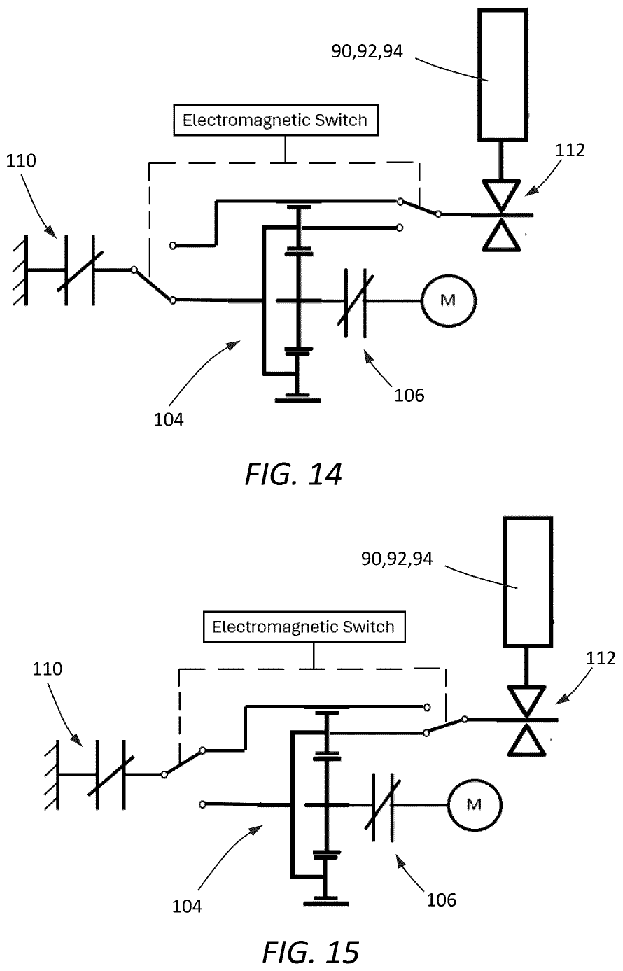

FIGS. 14 through 19 incorporate electromagnetic clutches 112 to enable gear ratio switching capable of changing output load capacity and rotational speed. The drive motor M drives one of the components (sun gear, ring gear, or carrier) of the planetary gear system 104 as input to the rotation actuation mechanism to articulate the finger joints 90, 92, and/or 94 of the fingers 14-22. Depending on the state of the electromagnetic clutches 112, different gear ratios and/or rotational directions are achieved by selecting which of the remaining components of the planetary gear system 104 serves as the rest gear component and output to the joint 90, 92, or 94 through the use of a stationary clutch 110 to transmit torque and angular motion to the finger 14, 16, 18, 20, or 22 to perform a task. In this manner, the electromagnetic clutches 112 enable smooth switching between different operational modes, resulting in a more universal, metamorphic, and versatile robotic hand 10.

When the drive motor M is directly connected to the sun gear of the planetary gear system 104 as shown in FIGS. 14 and 15, the electromagnetic clutch 112 is placed between the ring gear and the carrier. FIG. 14 depicts the electromagnetic clutch 112 as not activated, such that the ring gear is connected to the finger joint 90, 92, or 94 through the electromagnetic clutch 112 while the carrier is connected to a base through a stationary clutch 110. FIG. 15 depicts the electromagnetic clutch 112 as activated, such that the carrier is connected to the finger joint 90, 92, or 94 through the clutch 112 while the ring gear is connected to the base through the stationary clutch 110.

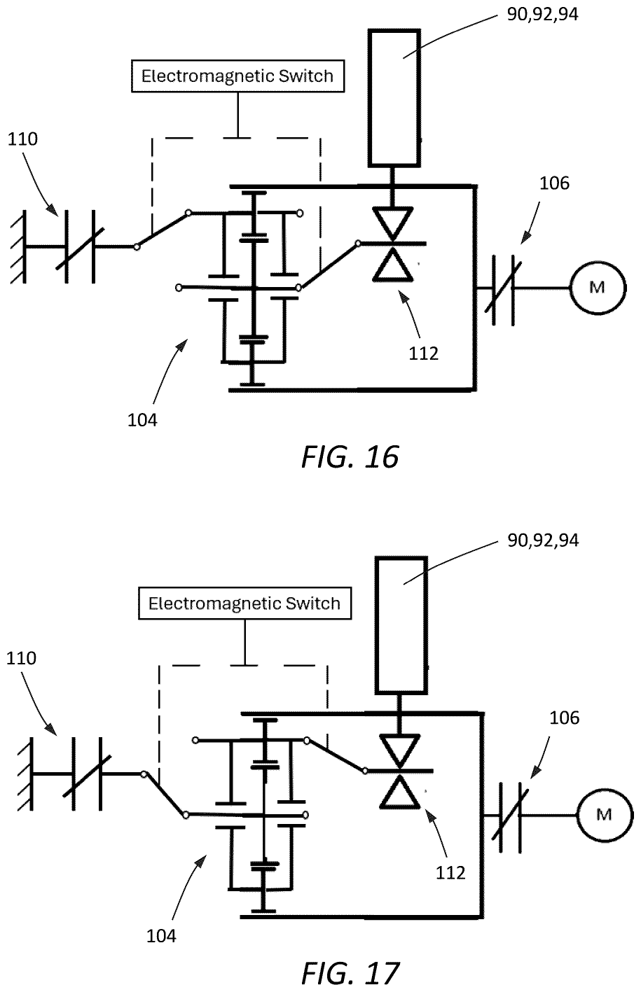

When the drive motor M is directly connected to the ring gear as shown in FIGS. 16 and 17, the electromagnetic clutch 112 is placed between the sun gear and carrier. FIG. 16 shows the electromagnetic clutch 112 as not activated, such that the sun gear is connected to the finger joint 90, 92, or 94 through the clutch 112 while the carrier is connected to a base through a stationary clutch 110. When the electromagnetic clutch 112 is activated as shown in FIG. 17, the carrier is connected to the finger joint 90, 92, or 94 through the clutch 112 while the sun gear is connected to the base through the stationary clutch 110.

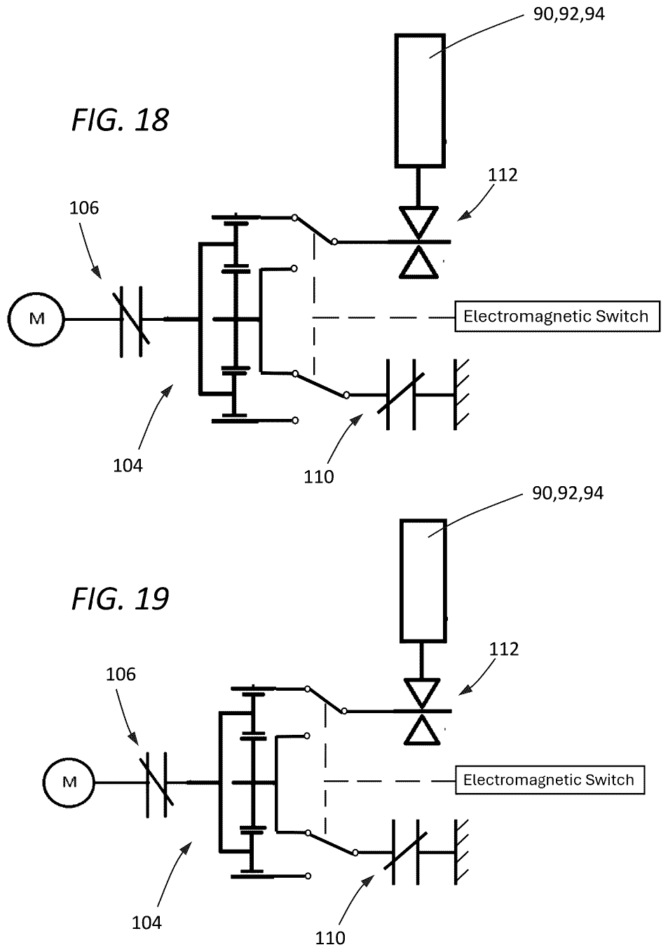

When the drive motor M is directly connected to carrier assembly as shown in FIGS. 18 and 19, the electromagnetic clutch 112 is placed between the ring gear and sun gear. FIG. 18 shows the electromagnetic clutch 112 as not active, such that the ring gear is connected to the finger joint 90, 92, or 94 through the clutch 112 while the sun gear is connected to a base through a stationary clutch 110. FIG. 19 shows the electromagnetic clutch 112 as active, such that the ring gear is connected to the finger joint 90, 92, or 94 through the clutch 112 while the sun gear is connected to the base through the stationary clutch 110.

A computer control system 200 (FIG. 1) may be provided for controlling actuation of the various linear actuators 30, 32, and 34 and rotation actuation mechanisms of the robotic hand 10. The control system 200 preferably includes both hardware components (e.g., electronic memory and processor) and software components executed by the hardware components, which execute instructions for controlling the functioning of the robotic hand 10 in aspects of motion control, path planning, inverse kinematics, and other related movement and/or gripping functions. The control system 200 may also incorporate various sensors to sense, for example, speed, direction, force, and/or proximity, which can provide status information to the control system 200 and subsequently used by the control system 200 to control movements of the robotic hand 10.

The robotic hand 10 disclosed herein provides a universal robotic approach, using a single device with several key features in mechanical structure for its functionality and performance. The structure of the palm frame 12 provides the robotic hand 10 with structural versatility, which allows it to adjust to many various structural configurations tailored to accommodate diverse needs and tasks. By controlling the amount of extension or retractions of the linear actuators 30, 32, and 34 (“prismatic joints”) of the palm frame 12, the palm frame 12 can be scaled and deformed to meet many different form factors configurations. This provides a convenient way for the articulating fingers 14-22 to achieve the best enveloping grasp effect when grabbing objects, thereby increasing the stability of the grasp. The finger joints 90, 92, and 94 of the robotic hand 10 have a bi-directional rotation design such that the palm frame 12 has a bi-directional grasping capability. This allows the robotic hand 10 to function as either/both a left hand and/or right hand.

The robotic hand 10 has several advantages over conventional robotic hands. For example, a single universal robotic hand 10 can provide sufficient structural versatility using a single device that yields various structural scales, tailored to accommodate diverse needs and tasks. The range of motion of the robotic hand 10 has benefits of bi-directional motion design so that it has double workspace or be universal for left hand/right hand applications. The controllable scales and grasps using 2-dimensional palm mechanisms for various workspace and trajectories of operations. The control system 200 for the robotic hand 10 in terms of both hardware (e.g., computers hardware and/or actuator hardware, such as hydraulics, pneumatics, gears, motors, etc.) and software can be used for various aspects of motion control, path planning, inverse kinematics, and related functions and movements of the components of the robotic hand 10. The robotic hand 10 yields improved adaptability for different applications and can be used in various diverse application domains, such as industrial, medical, and service sectors. The robotic hand 10 makes differences in aspects of mechanical structure, mechanisms, different sensing and actuating, motion control, safety, etc., possible.

A robotic hand according to the principles disclosed herein may be useful in many different applications and scenarios, such as a smart prosthetic hand implant, and industrial robotic hand, for medical procedures, in service sectors, and essentially any application where grasping and manipulating objects is needed. Further, although the robotic hand 10 it typically conceived of as having a size generally on the scale of a typical adult human hand (e.g., about 15-35 cm across from thumb tip to little finger tip), the robotic hand 10 could be produced at any necessary size, from smaller, for example for very fine applications on scale of millimeters or micrometers, to much larger, for example for large industrial applications on the scale of meters, the only limitations being the ability to manufacture components to a desired smaller or larger size. In some embodiments, the robotic hand 10 provides a dexterous and universal prosthetic hand. One universal device of the robotic hand 10 can function as either (or both) a left hand or right hand of variable sizes and shapes according to the needs of a particular situation. In some embodiments, the robotic hand 10 provides a dexterous and universal manipulator, which can find its applications in industrial operations to meet the needs of different sizes and shapes without retooling.

The robotic hand 10 may be made of any material suitable for providing sufficient stiffness and strength for a given design use. Some examples include metals, plastics, ceramics, wood, and/or composites.

In some applications, the robotic hand 10 may include a flexible outer skin that covers some or all of the palm frame 12 and finger components, as well as any motion control and/or actuation components for articulating the articulating fingers 14-22 and/or for adjusting the size and/or shape of the palm frame 12. The outer skin may be a flexible material or a jointed stiff material. Such an outer skin may be formed of any material suitable for a given design use. Some examples include silicon rubber, plastic, rubber, cloth, metal, and/or composites.

As previously noted above, though the foregoing detailed description describes certain aspects of one or more particular embodiments of the invention, alternatives could be adopted by one skilled in the art. For example, the robotic hand 10 and its components could differ in appearance and construction from the embodiments described herein and shown in the drawings, functions of certain components of the robotic hand 10 could be performed by components of different construction but capable of a similar (though not necessarily equivalent) function, and various materials could be used in the fabrication of the robotic hand 10 and/or its components. As such, and again as was previously noted, it should be understood that the invention is not necessarily limited to any particular embodiment described herein or illustrated in the drawings.

1. A robotic hand comprising
a palm frame comprising a first linear actuator comprising a first component and a second component that is linearly shiftable relative to the first component along a first actuation axis, a second linear actuator comprising a third component and a fourth component that is linearly shiftable relative to the first component along a second actuation axis, a third linear actuator comprising a fifth component and a sixth component that is linearly shiftable relative to the first component along a third actuation axis, and a link having a first end and a second end defining a fourth axis, wherein the second component is rotatably connected to the third component about a first rotational axis, the fourth component is rotatably connected to the fifth component about a second rotational axis, the sixth component is rotatably connected to the first end of the link about a third rotational axis, the second end of the link is rotatably connected to the first component about a fourth rotational axis, and each of the first axis, the second axis, the third axis, and the fourth axis are parallel with each other; and
a plurality of articulating fingers coupled to the second and third linear actuators of the palm frame;
wherein extending and/or retracting any one or more of the first linear actuator, the second linear actuator, and the third linear actuator changes the size of the palm within a palm plane and/or one or more angles about the first, second, and third rotational axes.

2. The robotic hand of claim 1, wherein the plurality of articulating fingers comprises a first jointed finger pivotably mounted to the third component such that the first jointed finger pivots about the second actuation axis, wherein the first jointed finger comprises at least two articulating links that articulate in a first finger plane, wherein the first finger plane is disposed at an acute angle relative to the second actuation axis.

3. The robotic hand of claim 1, wherein the plurality of articulating fingers comprises a second jointed finger mounted to the fifth component, wherein the second jointed finger comprises at least two articulating links that articulate in a second finger plane, wherein the second finger plane is parallel with the second actuation axis.

4. The robotic hand of claim 1, wherein the plurality of articulating fingers comprises a third jointed finger mounted to the fifth component, wherein the third jointed finger comprises at least two articulating links that articulate in a third finger plane, wherein the third finger plane is parallel with the second actuation axis.

5. The robotic hand of claim 1, wherein the plurality of articulating fingers comprises a fourth jointed finger pivotably mounted to the sixth component such that the fourth jointed finger pivots about the third actuation axis, wherein the fourth jointed finger comprises at least two articulating links that articulate in a fourth finger plane, wherein the fourth finger plane is disposed at an acute angle relative to the third actuation axis.

6. The robotic hand of claim 1, wherein the plurality of articulating fingers comprises a fifth jointed finger pivotably mounted to the sixth component such that the fifth jointed finger pivots about the third actuation axis, wherein the fifth jointed finger comprises at least two articulating links that articulate in a fifth finger plane, wherein the fifth finger plane is disposed at an acute angle relative to the third actuation axis.

7. The robotic hand of claim 1, wherein each of the plurality of articulating fingers articulates in a first direction toward a first side of the palm plane and in a second direction toward a second side of the palm plane.

8. The robotic hand of claim 1, wherein at least one of the first linear actuator, the second linear actuator, and third linear actuator comprises at least one of a hydraulic actuator, a pneumatic actuator, a rack and pinion actuator, and a servo motor.

9. The robotic hand of claim 1, the robotic hand further comprising rotation actuation mechanisms that individually actuate and control articulation of each of the articulating fingers.

10. The robotic hand of claim 9, wherein at least one of the rotation actuation mechanisms comprise at least one of a hydraulic actuator, a pneumatic actuator, a geared actuator, a servo motor, and a linear actuator.

11. The robotic hand of claim 9, wherein at least a first rotation mechanism of the rotation actuation mechanisms comprises a drive motor coupled to a planetary gear system having at least one component connected to a finger joint of one of the actuating fingers.

12. The robotic hand of claim 11, wherein the first rotation actuation mechanism further comprises an electromagnetic clutch adapted to selectively connect the finger joint to different components of the planetary gear system.

13. A method of using the robotic hand of claim 1, the method comprising:
changing the size of the palm and/or one or more angles about the first, second, and third axes within the palm plane by extending and/or retracting any one or more of the first linear actuator, the second linear actuator, and the third linear actuator.

14. A robotic hand comprising:
a palm frame; and
a plurality of articulating fingers coupled to the palm frame, each of the plurality of articulating fingers articulating in a first direction toward a first side of the palm plane and in a second direction toward a second side of the palm plane.

15. The robotic hand of claim 14, wherein the plurality of articulating fingers comprises:
a first articulating finger that articulates about a first component of the palm frame;
a second articulating finger that articulates about a second component of the palm frame; and
a third articulating finger that articulates about a third component of the palm frame;
wherein at least two of the first component, the second component, and the third component are disposed at an acute angle relative to each other; and
wherein each of the first articulating finger, the second articulating finger, and the third articulating finger can articulate toward each other on the first side of the palm frame and can articulate toward each other on the second side of the palm frame.

16. The robotic hand of claim 15, wherein the plurality of articulating fingers comprises a fourth articulating finger that articulates about the second component and can articulate toward each of the first side and the second side of the palm frame.

17. The robotic hand claim 16, wherein the plurality of articulating fingers comprises a fifth articulating finger that articulates about the third component and can articulate toward each of the first side and the second side of the palm frame.

18. The robotic hand of claim 14, wherein the palm frame comprises first, second, and third linear actuators, the first, second, and third linear actuators defining a palm plane, and wherein extending and/or retracting any one or more of the first, second, and third linear actuators changes the size of the palm and/or one or more angles about the first, second, and third axes within the palm plane.

19. The robotic hand of claim 14, the robotic hand further comprising rotation actuation mechanisms that individually actuate and control articulation of each of the articulating fingers, at least a first rotation mechanism of the rotation actuation mechanisms comprising a drive motor coupled to a planetary gear system having at least one component connected to a finger joint of one of the actuating fingers and an electromagnetic clutch adapted to selectively connect the finger joint to different components of the planetary gear system.

20. A method of using the robotic hand of claim 14, the method comprising articulating the articulating fingers together toward a first side of the palm plane and articulating the fingers together toward a second side of the palm plane.
